(19) United States
(12) Patent Application Publication
(10) Pub. No.: US 2016/0032398 A1
(43) Pub. Date: Feb. 4, 2016

(54) METHODS FOR TREATING CANCER

(71) Applicant: COMBIMMUNE, INC., Palo Alto, CA (US)

(72) Inventors: Russell K. Pachynski, San Francisco, CA (US); Holbrook E. Kohrt, San Francisco, CA (US); Jason Yonehiro, San Jose, CA (US)

(21) Appl. No.: 14/776,301

(22) PCT Filed: Mar. 17, 2014
(86) PCT No.: PCT/US14/30676

§ 371 Date: Sep. 14, 2015

Related U.S. Application Data
(60) Provisional application No. 61/794,182, filed on Mar. 15, 2013.

(51) Int. Cl. C12Q 1/68; A61K 45/06; A61K 39/395; A61K 38/17

(52) U.S. Cl. CPC C12Q 1/6886; A61K 38/177; A61K 45/06; A61K 39/3955; C12Q 2600/136

(57) ABSTRACT

The disclosure features a method of treating cancer by lowering a patient's two gene score (TGS), particularly by increasing the number of tumor-infiltrating leukocytes. In addition, a TGS animal model and uses thereof are provided.

### CROSS REFERENCE TO RELATED APPLICATIONS

This application claims priority to U.S. Provisional Application No. 61/794,182 filed on Mar. 15, 2013, which application is incorporated by reference herein in its entirety.

### FIELD OF THE INVENTION

The present disclosure relates generally to a method of treating cancer by increasing the number of tumor-infiltrating leukocytes and/or improving a marker-based prognostic score. The present invention further relates to animal models for screening for compounds that affect such marker-based prognostic scores and methods of use thereof.

### BACKGROUND OF THE INVENTION

Non-Hodgkin lymphoma (NHL) represents a significant medical burden with approximately 130,000 new cases worldwide annually, the majority of which are incurable today. Diffuse large B-cell lymphoma (DLBCL) is the most common subtype of NHL, accounting for about 30-35% of total cases. Although commonly used first-line regimens such as R-CHOP (rituximab, cyclophosphamide, doxorubicin, vincristine, prednisone) can result in high initial complete remission rates in DLBCL patients, many of these patient have recurrences, with about 30-40% of patients eventually dying from the disease (Coiffier et al. N Engl J Med 346: 235-242, 2002). Thus, accurately predicting who will respond well and ultimately survive, and being able to distinguish those patients from others who are at highest risk for disease relapse and death is very important.

The current system of risk stratification that is the standard of care for DLBCL patients is the International Prognostic Index (IPI) (A predictive model for aggressive non-Hodgkin's lymphoma. The International Non-Hodgkin's Lymphoma Prognostic Factors Project. N Engl J Med 329: 987-994, 1993). The IPI consists of a composite score utilizing the following clinical factors: age, stage of disease, number of extra-nodal sites involved, serum lactose dehydrogenase (LDH) level, and performance status. This system of classification has been used in clinical trials as the main mechanism for risk stratification since its development. However, the IPI was developed in 1993 prior to the standard use of rituximab in DLBCL treatment, which had a significant impact on survival of DLBCL patients. Sehn et al. (Blood 109: 1857-1861, 2007) analyzed the performance characteristics of the IPI in the modern age of RCHOP therapy and showed that the IPI remained predictive, but only stratified patients into two risk groups. They also found that the IPI was unable to identify a group of patients with a risk of survival less than 50% (Sehn et al. Blood 109: 1857-1861, 2007). Additionally, the IPI does not fully represent the spectrum and heterogeneity of the disease (Zelenetz et al. J Natl Compr Canc Netw 8: 288-334, 2010). This strongly suggests the need for better prognostic tools to more accurately stratify patient risk.

Ongoing studies are evaluating the efficacy of RCHOP compared with other more aggressive regimens in first line DLBCL. Other studies are continuing to look at the impact of autologous bone marrow transplantation on survival and outcomes in refractory or relapsed DLBCL. In order to better and more accurately stratify patients that are at highest risk of poor outcomes (e.g., refractory disease, recurrence or relapse), a two gene score prognostic test is described in US Patent Application Publication No. 2012/0134986, incorporated herein in its entirety. There is currently a need to identify patients that are potentially in need of more aggressive therapies or transplants, while sparing those patients with low risk of relapse or death. Satisfying this need would ultimately lead to personalized, tailored, and ultimately optimized, treatment plans, thereby minimizing overtreatment and maximizing aggressive therapy for those at highest risk.

Therapies for DLBCL have improved with the advent of rituximab, which was added to standard CHOP therapy. RCHOP has been the standard of care and first line treatment for the majority of patients with DLBCL, inducing high rates of initial response. However, long-term survival for these patients range from 30-50% (Pfreundschuh et al. Lancet Oncol 7: 379-391, 2006; Feugier et al. J Clin Oncol 23: 4117-4126, 2005; Coiffier Semin Oncol 29: 18-22, 2002), indicating that more efficacious treatments are needed. The addition of rituximab, an immunomodulatory monoclonal antibody, significantly improved both progression-free and overall survival in two pivotal clinical trials (Pfreundschuh et al. Lancet Oncol 7: 379-391, 2006 and Coiffier Semin Oncol 29: 18-22, 2002). This suggests that adding an immunotherapeutic agent to an existing regimen can significantly improve outcomes. Still, there remains a need to correlate ongoing methods of prognosticating risks to treatments that improve these prognostic markers.

### SUMMARY OF THE INVENTION

The present disclosure addresses these needs by providing a method of using a diagnostic test that more accurately risk stratifies DLBCL patients than the IPI in combination with therapies that can alter this diagnostic test readout, and, further provides a novel animal model for said diagnostic. This disclosure features methods of treating cancer, e.g., non-Hodgkin lymphoma, by increasing the number of tumor-infiltrating leukocytes and/or improving a marker-based prognostic score. In particular, the methods of the invention decrease a patient's two gene score (TGS), which is a prognostic algorithm for assessing risk in lymphoma involving LMO2, a gene expressed by a malignant B cell, and CD137 (TNFRSF9), a gene expressed by infiltrating leukocytes including T cells and natural killer (NK) cells.

One aspect provides a method of treating a patient diagnosed with a malignant tumor and having a first two gene score (TGS), wherein the first TGS indicates the patient is high risk, intermediate risk, or low risk, the method comprising administering a therapeutic that leads to an increased number of tumor-infiltrating leukocytes. Other maker based prognostic assays may also be used, for example assays that measure and correlate other markers from tumor cells versus infiltrating leukocytes. In one embodiment, the malignant tumor is a lymphoma or an adenocarcinoma. In a related embodiment, the lymphoma is a non-Hodgkin lymphoma. In a further embodiment, the non-Hodgkin lymphoma is diffuse large B-cell lymphoma (DLBCL). In another related embodiment, the adenocarcinoma is a breast adenocarcinoma, a colon adenocarcinoma, or a lung adenocarcinoma.

In another embodiment, the therapeutic is an immunomodulator. In one embodiment, the immunomodulator is a chemoattractant, an antibody, a cytokine, a chemokine, a small molecule, or a kinase inhibitor. In a particular embodiment, the chemoattractant is chemerin. In yet another embodiment, the therapeutic is administered locally. In one embodiment, the therapeutic is administered via intratumor injection.

In one embodiment, the administering lowers the first TGS of the patient, thereby providing a second TGS. In a further embodiment, the first TGS indicates the patient is high risk and the second TGS indicates the patient is intermediate risk or low risk. In another embodiment, the first TGS indicates the patient is intermediate risk and the second TGS indicates the patient is low risk.

In another embodiment, the method further comprises administering one or more chemotherapeutic agents to the patient. In a particular embodiment, the one or more chemotherapeutic agents comprise rituximab, cyclophosphamide, doxorubicin, vincristine, and prednisone (R-CHOP).

In one embodiment, an International Prognostic Index (IPI) score is combined with the first TGS to indicate risk. In another embodiment, the tumor-infiltrating leukocytes express CD137. In a particular embodiment, the tumor-infiltrating leukocytes are NK cells or T cells.

In another aspect, a method of modeling TGS in a non-human animal is provided, wherein the method comprises: obtaining a first sample from a tumor in a non-human animal, determining a TGS, observing a clinical outcome of the non-human animal, and correlating the clinical outcome with the TGS, thereby producing a TGS animal model. In one embodiment, the method further includes a step of classifying risk groups based on TGS. In another embodiment, the method further comprises the following steps: administering a therapeutic to the non-human animal, obtaining a second sample from the tumor after administering the therapeutic, determining a second TGS, comparing the first TGS and the second TGS, and correlating the change in TGS with the clinical outcome.

In one embodiment, the non-human animal is a mouse, a rat, a rabbit, a primate, a dog, or a pig. In another embodiment, the tumor is induced, spontaneous, or transplanted. In yet another embodiment, the species origin of the tumor differs from the non-human animal. In one embodiment, the determining step comprises measuring LMO2 and TNFRSF9 expression by RT-PCR.

In another embodiment, the therapeutic is an immunomodulator. In one embodiment, the immunomodulator is a chemoattractant, an antibody, a cytokine, a chemokine, or a kinase inhibitor. In a particular embodiment, the chemoattractant is chemerin. In one embodiment, the therapeutic is administered locally. In a particular embodiment, the therapeutic is administered via intratumor injection.

Another aspect provides a method of identifying a composition that lowers a TGS that comprises the steps of: obtaining a first sample from a tumor in a non-human animal, contacting the tumor in vivo with a composition, obtaining a second sample from the tumor, determining the TGS of the first sample and the second sample, wherein the composition lowers the TGS of the tumor if the TGS of the second sample is less than the TGS of the first sample. In one embodiment, the non-human animal is a mouse, a rat, a rabbit, a primate, a dog, or a pig. In another embodiment, the tumor is induced, spontaneous, or transplanted. In a particular embodiment, the species origin of the tumor differs from the non-human animal.

In one embodiment, the determining step comprises measuring LMO2 and TNFRSF9 expression by RT-PCR. In another embodiment, the method further comprises a step of correlating TGS and clinical outcome. In one embodiment, the composition is an immunomodulator.

### BRIEF DESCRIPTION OF THE DRAWINGS

FIG. 1 is a scatter plot that shows that LMO2 and TNFRSF9 are among the best univariate predictors of survival considering all measured genes in both R-CHOP and CHOP treated lymphoma patients.

FIG. 2 is a series of line graphs that shows the stratification of clinical outcomes based on level of expression of TNFRSF9 in patients with adenocarcinomas of the breast (left panel), colon (middle panel), and lung (right panel). This data indicates that TNFRSF9 can be used as a predictive marker in multiple tumor types.

FIG. 3 is a box plot that shows the measured expression level of TNFRSF9 in multiple tumor types, thereby indicating its use across a wide variety of malignancies.

FIG. 4 is a line graph that shows the performance of the TGS comprising expression of LMO2 and TNFRSF9 in a cohort of lymphoma patients.

FIG. 5 is a line graph that shows the performance of the composite risk score integrating TGS and IPI (TGS-IPI) in a cohort of lymphoma patients.

FIG. 6 is a series of line graphs that shows that the TGS can further stratify individual IPI risk categories (low, intermediate, and high) of DLBCL patients treated with RCHOP or CHOP, indicating that it is more accurate than the IPI alone in predicting clinical outcomes in lymphoma patients.

FIG. 7 is a dot plot and histogram that shows that CD137 (TNFRSF9) is present on leukocytes, specifically T cells and NK cells.

FIG. 8 is a bar graph that shows that NK and T cells in murine tumors increased with treatment by an immunomodulatory agent, chemerin. This shows that treatment of tumors can result in the increased representation of leukocytes in tumors.

FIG. 9 is a bar graph that shows that treatment of tumors with chemerin resulted in increases in leukocyte populations in the tumor, specifically NK and T cells.

FIG. 10 is a line graph that shows the clinical benefit of an immunomodulatory agent, chemerin, and the dependence of this on NK cells. This indicates that the presence of NK cells in the tumor is required for clinical benefit.

FIG. 11 is a line graph that shows tumor size of chemerin secreting B16 melanoma tumors (RARRES) and control B16 melanoma tumors in mice.

FIG. 12 is a bar graph that shows relative expression of LMO2 and CD137 in control and chemerin secreting B16 melanoma tumors.

FIG. 13A is a bar graph that shows the percentage of tumor-infiltrating leukocytes (TIL) in B16 melanoma tumors treated with cetuximab, EGFR-expressing B16 melanoma tumors treated with cetuximab, and chemerin secreting B16 melanoma tumors. Data is from 11 days post implant. Groups that received cetuximab were treated on Day 9.

FIG. 13B is a bar graph that shows the percentage of Total T cells, CD8+ T cells anad NK cells of the CD45+ tumor-infiltrating leukocytes (TIL) in B16 melanoma tumors treated with cetuximab, EGFR-expressing B16 melanoma tumors treated with cetuximab, and chemerin secreting B16 melanoma tumors.

FIG. 14 is a bar graph that shows the measured CD137 expression on total tumor-infiltrating leukocytes in B16 melanoma tumors treated with cetuximab, EGFR-expressing B16 melanoma tumors treated with cetuximab, and chemerin secreting B16 melanoma tumors 48 hours after cetuximab treatment. n=3 mice per group. FACS shown on CD45+ tumor-infiltrating leukocytes.

FIG. 15 is a bar graph that shows the measured CD137 expression on CD8+ T cells and NK cells in B16 melanoma tumors treated with cetuximab, EGFR-expressing B16 melanoma tumors treated with cetuximab, and chemerin secreting B16 melanoma tumors.

FIG. 16 is a bar graph that shows the tumor size of the B16 melanoma tumors treated with cetuximab, EGFR-expressing B16 melanoma tumors treated with cetuximab, and chemerin secreting B16 melanoma tumors as measured on Day 4 and Day 11.

FIG. 17A is a bar graph that shows relative LMO2 expression at 48 hours post treatment, and FIG. 17B is a bar graph that shows relative CD137 expression at 48 hours post treatment.

FIG. 18A is a bar graph that shows relative LMO2 expression pretreatment, at 2 days post treatment, and at 8 days post treatment, and FIG. 18B is a bar graph that shows relative CD137 expression pretreatment, at 2 days post treatment, and at 8 days post treatment.

FIG. 19 is a bar graph that shows the ratio of relative expression levels of CD137 to LMO2 pretreatment, at 2 days post treatment, and at 8 days post treatment.

FIG. 20 is a series of histograms that show representative CD137 expression on CD3-DX5+NK cells isolated from the spleen, peripheral blood, or tumor analyzed at 24, 72, or 168 hours following cetuximab treatment or isotype control in EGFR-expressing SCC6 tumor-bearing nu/nu mice (n=3 mice per group).

FIG. 21A is a schema that BALB/c mice were inoculated with 1×10⁶ TUBO-EGFR tumor cells subcutaneously on the left flank and that cetuximab and/or anti-CD137 mAb was administered starting Day 14 post tumor inoculation.

FIG. 21B is a line graph that shows tumor size in mice that received Rat IgG control on Day 14 (), cetuximab on Day 14 (▪), anti-CD137 antibody on Day 15 (♦), or cetuximab on Day 14 and anti-CD137 antibody on Day 15 (▴) with each injection repeated weekly for 4 total injections (*p=0.047).

FIG. 21C is a line graph that shows overall survival of BALB/c mice inoculated with TUBO-EGFR tumor cells and received cetuximab and/or anti-CD137 mAb (*p<0.001).

FIG. 22A is a bar graph that shows the percentage of total CD45+ leukocytes that are CD137+ at Day 7 of an in vitro co-culture of splenocytes and EGFR-expressing B16 melanoma cells. FIG. 22B is a bar graph that shows the percentage of total NK cells that are CD137+ at Day 7 of the in vitro co-culture.

FIG. 23 is a bar graph that shows the percentage of dead CD45-cells at 24 hours and Day 7 of an in vitro co-culture of splenocytes and EGFR-expressing B16 melanoma cells.

### DETAILED DESCRIPTION OF THE INVENTION

The compositions and methods featured herein relate to prognostic, diagnostic and treatment methods for cancer. The present invention provides methods of treating cancer by increasing the number of tumor-infiltrating leukocytes wherein such treatment methods may be correlated to improvements in a marker-based prognostic score. In some embodiments, the present disclosure provides for the use of the two gene score (TGS) diagnostic test in 1) an animal model and 2) a treatment setting in humans. The TGS was initially developed and described by Alizadeh et al. (Blood 118: 1350-1358, 2011). The TGS is a score calculated by using measured levels of two genes in a tumor sample, LMO2 and TNFRSF9. TNFRSF9 is also known as CD137 and can be found on NK cells, T cells, and other leukocytes. LMO2 is a transcription factor found in tumor cells. A biopsy sample is obtained, and the expression levels of TNFRSF9 and LMO2 are determined. The relative quantity of each gene transcript is then used to calculate the TGS using the following formula: TGS=(−0.32×LMO2)+(−0.29×TNFRSF9). The score is then used alone or in combination with the International Prognostic Index (IPI) and stratifies lymphoma patients into different risk groups, and more accurately predicts clinical outcomes and survival in lymphoma patients compared to the IPI alone.

Both LMO2 and TNFRSF9 are found in many different tumor types. TNFRSF9 (CD137) is an independent prognostic factor in several tumor types in addition to lymphoma, and higher expression levels correlate with better clinical outcomes. Levels of TNFRSF9 RNA measured in tumors correlates with the amount of surface CD137 on cells in the tumor biopsy sample. CD137 expression is not seen on tumor cells, but rather leukocytes within the tumor sample. Thus, the TGS enables one to monitor both the tumor (via LMO2) and the immune cells (via TNFRSF9) in the tumor biopsy and gives valuable information about the not only the representation of the immune system within the tumor (TNFRSF9), but also the clinical outcomes of the patient. Prognostic scores that employ other tumor markers and other immune cell markers may be developed and used in the methods of the invention.

The present disclosure provides for 1) the TGS to be modeled in animals, for both prediction of outcomes as well as evaluating interventions and 2) the selection of patients with a certain TGS to identify them for a particular treatment or therapy, which may further impact their TGS if evaluated after said intervention. The methods described herein allow one to effectively select patients with malignancies and monitor their therapeutic responses using the TGS.

### DEFINITIONS

As used in this specification and the appended claims, the singular forms “a,” “an” and “the” include plural references unless the content clearly dictates otherwise.

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art.

The terms “two gene score” and “TGS,” are used interchangeably herein and refer to a diagnostic test that incorporates LMO2 and TNFRSF9 gene expression. TGS is described in greater detail by Alizadeh et al. (Blood 118:1350-1358, 2011) and in US Patent Application Publication No. 2012/0134986. TGS can be used to stratify lymphoma patients into risk categories. Higher expression of LMO2 and TNFRSF9 corresponds with reduced patient risk, and results in a lower TGS score. Accordingly, a low TGS score corresponds with a lower patient risk, while a higher TGS score corresponds with a higher risk. The TGS can be integrated with the International Prognostic Index (IPI) to provide a composite score, referred to as “TGS-IPI” herein.

TGS is calculated using the following formula: TGS=(−0.32×LMO2)+(−0.29×TNFRSF9), where LMO2 and TNFRSF9 represent the values for the expression level of the respective gene. The following is an alternative formula for calculating a TGS value: TGS=−LMO2−TNFRSF9.

TGS-IPI is calculated using the following formula: TGS-IPI=(0.93×TGS)+(0.6×IPI)+4. The following is an alternative formula for calculating a TGS-IPI value: TGS-IPI=2×IPI−LMO2−TNFRSF9.

The terms “patient risk,” “risk category,” and “risk group” are used interchangeably and refer to patient prognosis and predicted clinical outcome. Accordingly, the lower chances of overall survival a patient has, the higher the patient's risk. A high risk patient may be at increased risk of cancer-related death, relapse, and/or poor response to treatment.

The term “high risk” refers to a low likelihood of overall survival with standard treatment (e.g., less than about a 60% chance of 2 year overall survival). The term “intermediate risk” refers to a moderate likelihood of overall survival with standard treatment (e.g., less than about an 80% chance of 2 year overall survival). The term “low risk” refers to a high likelihood of overall survival with standard treatment (e.g., greater than about a 90% chance of 2 year overall survival).

In particular, TGS and TGS-IPI can be used to stratify and indicate high, intermediate, and low risk groups that correspond with upper, middle, and lower tertiles, respectively. For example, in one embodiment a TGS greater than about −0.9 is associated with high risk, a TGS of about −1.6 to about −0.9 is associated with intermediate risk, and a TGS less than or equal to about −1.6 is associated with low risk. In another example, a TGS-IPI greater than about 4.5 is associated with high risk, a TGS-IPI of about 3.5 to about 4.5 is associated with intermediate risk, and a TGS-IPI of about 3.5 or below is associated with low risk.

The terms “tumor,” “malignancy,” “cancer,” and “proliferative disease or disorder” are used interchangeably herein and refer to malignant cell growth. Examples of tumors include, but are not limited to, lymphoma, non-Hodgkin lymphoma, diffuse large B cell lymphoma (DLBCL), adenocarcinoma (e.g., breast, colon, or lung), adenomas, carcinomas, squamous or basal cell tumors, nerve or central nervous cell tumors, and melanoma.

The term “administering,” as used herein, refers to any mode of transferring, delivering, introducing, or transporting a therapeutic, e.g., a pharmaceutical drug or other agent, to a patient or non-human animal. Such modes include oral administration, topical contact, intravenous, intraperitoneal, intramuscular, intranasal, or subcutaneous administration. The administration may be local (e.g., at or near the site of the tumor) or systemic.

The terms “therapeutic,” “therapy,” “treatment,” and “therapeutic agent” are used interchangeably and refer to an agent that, following administration, leads to a desired biological effect (e.g., leads to an increase in the number of tumor-infiltrating leukocytes, a reduction of tumor size, a slowing of tumor growth, or a decrease in TGS). In certain embodiments, the therapeutic is an immunomodulator. Examples of therapeutics include, but are not limited to, chemerin, monoclonal antibodies, polyclonal antibodies, antibody fragments, antibody fusion proteins, components of antibodies with or without other fused moieties that act to bind specific targets in or around tumors, cytokines, chemokines, small molecules, kinase inhibitors, cytotoxic chemotherapies, antibiotics, herbs, supplements, vitamins, foodstuffs, hormonal therapies, viral vectors, non-viral vectors, native or modified nucleic acids, peptides or polypeptides.

The term “antibody” (Ab) as used herein includes monoclonal antibodies, polyclonal antibodies, multispecific antibodies (e.g., bispecific antibodies), and antigen-binding fragments thereof. The term “immunoglobulin” (Ig) is used interchangeably with “antibody” herein. Examples of antibodies include rituximab and anti-CD137.

The terms “polypeptide,” “protein” and “peptide” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “tumor-infiltrating leukocytes” (TILs) refers to leukocytes present in the tumor microenvironment. Tumor-infiltrating leukocytes can be examined in a tumor biopsy sample. Examples of tumor-infiltrating leukocytes are NK cells and T cells. In particular, tumor-infiltrating leukocytes include CD137+ cells.

As used herein, “treating” or “treatment” refers to an approach for obtaining beneficial or desired results, including and preferably clinical results. Treatment can involve optionally either the amelioration of symptoms of the disease or condition, or the delaying of the progression of the disease or condition.

As used herein, unless the context makes clear otherwise, “prevention,” and similar words such as “prevented,” “preventing” etc., indicates an approach for preventing, inhibiting, or reducing the likelihood of, the onset or recurrence of a disease or condition. It also refers to preventing, inhibiting, or reducing the likelihood of, the occurrence or recurrence of the symptoms of a disease or condition, or optionally an approach for delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, “inhibiting cell growth” or “inhibiting proliferation of cells” refers to reducing or halting the growth rate of cells. For example, by inhibiting the growth of tumor cells, the rate of increase in size of the tumor may slow. In other embodiments, the tumor may stay the same size or decrease in size, i.e., regress. In particular embodiments, the rate of cell growth or cell proliferation is inhibited by at least 20%, at least 30%, at least 40%, at least 50%, at least 60%, at least 70%, at least 80%, or at least 90%.

A “non-human animal” refers to any animal other than a human such as, e.g., avians, reptiles and mammals. “Non-human mammal” refers to an animal other than humans which belongs to the class Mammalia. Examples of non-human animals include, but are not limited to, non-human primates, rodents, mice, rats, rabbits, dogs, and pigs.

### TGS Animal Model

The present disclosure provides for the first time the establishment of an animal model of the TGS. This model is based on the human findings (Alizadeh et al. Blood 118:1350-1358, 2011) and recapitulates the human TGS scores and clinical outcomes, thus enabling one to utilize animals in place of humans to model different scenarios. The model may be used in a treatment or therapy setting by assessing the TGS prior to and after said treatment or therapy. This allows one to not only select those animals with particular TGS scores, but also evaluate the impact of a given treatment or therapy on the TGS.

Producing a TGS Animal Model

The non-human animal TGS model described herein recapitulates the findings in humans. In one embodiment, a method of modeling TGS in a non-human animal comprises obtaining a first sample from a tumor in a non-human animal, determining a TGS, observing a clinical outcome of the non-human animal, and correlating the clinical outcome with the TGS, thereby producing a TGS animal model.

The animals used to model TGS include, but are not limited to, mice, rats, rabbits, primates, dogs, pigs, etc. In particular embodiments, mice are used in the TGS model. For example, MYC transgenic mice develop a lymphoma similar to DLBCL. The model utilizes tumor-bearing animals. The tumors may be induced, spontaneous, or transplanted, either of origin of the same or different species. In one embodiment wherein the species origin of the tumor differs from that of the non-human animal (e.g., human tumor cells in a mouse), the non-human animal comprises an engineered immune system of the same species origin as the tumor.

Samples from the tumors are obtained, e.g., from a biopsy, and RNA or other nucleic acid is obtained using standard procedures. Gene expression levels may be determined or measured using any of a number of available techniques commonly known in the art including, e.g., polymerase chain reaction (PCR), reverse transcriptase-PCR (RT-PCR), real time quantitative PCR (qPCR), RT-qPCR, northern blot, and Southern blot. In one embodiment, the expression of LMO2 and TNFRSF9 is evaluated by RT-PCR.

The animal TGS is obtained by using methods equivalent to those used to obtain the TGS in humans, wherein LMO2 and TNFRSF9 levels are determined (e.g., by RT-PCR) and algorithms are used to calculate the score. Any one of a number of probes to LMO2 or TNFRSF9 can be used to determine expression level by RT-PCR. For example, RT-PCR can be performed using the following primers for the two genes: LMO2 (Hs00277106_m1) and TNFRSF9 (Hs00155512_m1). In addition, the polynucleotide sequences of LMO2 and TNFRSF9 are highly conserved across species.

A number of tumor-bearing animals are used and evaluated to calculate TGS, and the clinical outcomes are assessed. For example, survival rate, weight of the animal, tumor size, tumor growth rate, number of metastases, and size of metastases may be measured and recorded. The TGS scores and clinical outcomes are then correlated in order to establish risk groups. In a particular embodiment, high risk and low risk groups are established. In another embodiment, high risk, intermediate risk, and low risk groups are established. The stratified risk groups may have the same or different values as the human lymphoma risk groups as determined by TGS and/or TGS-IPI (Alizadeh et al. Blood 118: 1350-1358, 2011). The TGS non-human animal model enables one to evaluate an animal using the TGS and predict its clinical outcome, as one does in the human.

Evaluating Therapeutics Using the TGS Animal Model

The TGS animal model may be used to evaluate a therapeutic that modulates the TGS of the tumor and/or leads to an increase in the number of tumor-infiltrating leukocytes. With regard to modulating the TGS of the tumor, the therapeutic preferably lowers the TGS. Such a reduction in TGS may correlate with a decreased risk and increased the likelihood of successful treatment. It is also contemplated that the TGS animal model may be used in a clinical setting to analyze the effect of a therapeutic on a particular patient's tumor. For example, the tumor in the non-human animal may be derived from a biopsy.

In one embodiment, the method of modeling TGS in a non-human animal comprises obtaining a first sample from a tumor in a non-human animal, determining a TGS, administering a therapeutic to the non-human animal, obtaining a second sample from the tumor after administering the therapeutic, determining a second TGS, comparing the first TGS and the second TGS, and correlating the change in TGS with the clinical outcome. Preferably, the second TGS score is lower than the first TGS. It is contemplated that the reduction in TGS may place the animal into a lower risk category (e.g., from high risk to intermediate or low risk). In a particular embodiment, administering the therapeutic leads to an increase in the number of tumor-infiltrating leukocytes. The increase in tumor-infiltrating leukocytes, particularly NK cells and T cells, may raise the level of expression of TNFRSF9 in the tumor, thereby decreasing the TGS.

The therapeutic may be an immunomodulator, such as, a chemoattractant, an antibody, a cytokine, a chemokine, or a kinase inhibitor. In a particular embodiment, the chemoattractant is chemerin. It is contemplated that a therapeutic may be administered locally or systemically. For example, it may be preferable to locally administer a chemoattractant. One example of local administration is intratumor injection.

Another embodiment provides a method of identifying a composition that lowers a TGS that comprises obtaining a first sample from a tumor in a non-human animal, contacting the tumor with a composition, obtaining a second sample from the tumor, determining the TGS of the first sample and the second sample, wherein the composition lowers the TGS of the tumor if the TGS of the second sample is less than the TGS of the first sample. The composition may lower the TGS of the tumor by leading to an increase in the number of tumor-infiltrating leukocytes. In a particular embodiment, the tumor-infiltrating leukocytes are CD137+. In one embodiment, the composition comprises a novel therapeutic.

### TGS In Vitro Assay

The present disclosure provides a TGS assay that is performed in vitro. An in vitro TGS assay incorporates the main components from the tumor microenvironment used in determining a TGS, namely tumor cells and immune cells. The in vitro TGS assay may be used to, e.g., identify therapeutic agents or combinations thereof that are effective in lowering the TGS for a particular type of cancer or an individual patient diagnosed with a malignant tumor. The cells used in the in vitro TGS assay may be obtained from a human (e.g., a healthy donor or a patient diagnosed with a malignant tumor) or an animal (e.g., mouse, rat, rabbit, non-human primate). In one embodiment, the cells are obtained from an animal used to model the malignant tumor.

In one embodiment of the invention, an in vitro TGS assay comprises a co-culture of tumor cells and leukocytes. Examples of tumor cells include immortalized tumor cell lines and tumor cells obtained (e.g., biopsy sample) from a patient diagnosed with a malignant tumor. Examples of tumor types include, e.g., lymphoma, non-Hodgkin lymphoma, diffuse large B cell lymphoma (DLBCL), adenocarcinoma (e.g., breast, colon, or lung), adenomas, carcinomas, squamous or basal cell tumors, nerve or central nervous cell tumors, and melanoma. Leukocytes may be obtained from an animal (e.g., splenocytes or PBMCs) or a human (e.g. PBMCs). In one embodiment, the leukocytes are a cell line (e.g., human or non-human).

In one embodiment, the tumor cells and leukocytes (e.g., T cells and/or NK cells) are co-cultured for a period of about 24, 48, 72, 96, 120, 144, 168, or more hours. A therapeutic agent, or combination of therapeutic agents, is added to a test group in order to examine the effects of the therapeutic agent on the components of the TGS. At various time points, parameters such as, e.g., CD137 expression, LMO2 expression, tumor cell growth, leukocyte cell growth, and leukocyte activation markers may be measured. In this way, the effect a therapeutic agent will have on a tumor can be examined in vitro. Furthermore, it is contemplated that tumor cells and leukocytes from a patient diagnosed with a malignant tumor can be treated with one or more therapeutic agents in vitro in order to identify a treatment regimen and/or predict the patient's response to the tumor.

### Methods of Treatment

The present disclosure also provides for the first time the selection and treatment of a patient with a malignancy based on the TGS. A patient with a malignancy can be selected based on his or her TGS and undergoes treatment or therapy for the malignancy. Additionally, the re-evaluation of the TGS after said treatment or therapy may be performed as a readout or report of response to treatment or therapy. For example, the impact that a treatment or therapy has on the TGS or TGS-IPI can be determined, as well as show how an increase in leukocytes in the tumor(s) correlates with an altered TGS or TGS-IPI.

In one method of treatment, one selects a patient with a given TGS and uses this for a basis for treatment or therapy. The patient may have a lymphoma or another malignancy on which the TGS is performed, as there is evidence that TNFRSF9 and LMO2 are predictive of outcomes and disease state in malignancies other than lymphoma (Alizadeh et al. Blood 118: 1350-1358, 2011 and Ma et al. J Pathol 211: 278-285, 2007). One then treats the patient with the selected TGS with a treatment or therapy. Such treatments or therapies could be comprised of: chemerin, monoclonal antibodies, polyclonal antibodies, antibody fragments, antibody fusion proteins, components of antibodies with or without other fused moieties that act to bind specific targets in or around tumors, cytokines, chemokines, small molecules, kinase inhibitors, cytotoxic chemotherapies, antibiotics, herbs, supplements, vitamins, foodstuffs, hormonal therapies, viral and non-viral vectors, native or modified nucleic acids, peptides or polypeptides, etc., or any combinations of such.

Any of these treatments or therapies may increase the representation of leukocytes within the tumor in patients. Accordingly, one then re-evaluates the patient's TGS following administration of the therapeutic. In this manner, one selects patients having a certain TGS and administering to said patient a composition comprising any treatment or therapy that leads to increased numbers of leukocytes in the tumor, thus potentially altering their TGS.

In one embodiment, a method of treating a patient diagnosed with a malignant tumor comprises calculating a first TGS, administering a treatment regimen to the patient, calculating a second TGS, wherein the second TGS is lower than the first TGS, and continuing the treatment regimen. In another embodiment, a method of treating a patient diagnosed with a malignant tumor comprises calculating a first TGS, administering a therapeutic agent to the patient, calculating a second TGS, wherein the second TGS is lower than the first TGS, thereby indicating that the therapeutic agent is effective in treating the patient.

In one embodiment, a method of treating a patient diagnosed with a malignant tumor comprises calculating a first TGS, administering a treatment regimen to the patient, calculating a second TGS, wherein the second TGS is greater than or equal to the first TGS, and discontinuing the treatment regimen. In another embodiment, a method of treating a patient diagnosed with a malignant tumor comprises calculating a first TGS, administering a therapeutic agent to the patient, calculating a second TGS, wherein the second TGS is greater than or equal to the first TGS, thereby indicating that the therapeutic agent is not effective in treating the patient.

In another embodiment, a method of treating a patient diagnosed with a malignant tumor comprises calculating a first TGS, administering a treatment regimen to the patient, calculating a second TGS, wherein the second TGS is greater than or equal to the first TGS, and administering a different treatment regimen. In one embodiment, a method of treating a patient diagnosed with a malignant tumor comprises calculating a first TGS, administering a treatment regimen to the patient, calculating a second TGS, wherein the second TGS is greater than or equal to the first TGS, and modifying the treatment regimen. Modifying the treatment regimen may be accomplished, e.g., by increasing the dosage and/or frequency of administering one or more therapeutic agents of the treatment regimen or adding one or more additional therapeutic agents to the treatment regimen.

In one embodiment, the first TGS is calculated from a tumor sample (e.g., biopsy) obtained before administering a therapeutic agent to the patient. In another embodiment, the first TGS is calculated from a tumor sample obtained on the same day as administering a therapeutic agent to the patient.

In one embodiment, the second TGS is calculated from a tumor sample obtained 1, 2, 3, 4, 5, 6, 7, 8, 9, 10, 11, 12, 13, 14 15, 16, 17, 18, 19, 20, or 21 or more days after administering the therapeutic agent to the patient. In one embodiment, the second TGS is calculated from a tumor sample obtained 7 to 14 days after administering the therapeutic agent to the patient.

In one embodiment, a method of treating a patient diagnosed with a malignant tumor and having a first two gene score (TGS), wherein the first TGS indicates the patient is high risk, intermediate risk, or low risk, comprises administering a therapeutic that leads to an increased number of tumor-infiltrating leukocytes. In one embodiment, the malignant tumor is a lymphoma (e.g., NHL or DLBCL) or an adenocarcinoma (e.g., breast, colon, or lung).

In one embodiment, a treatment regimen is selected for a patient diagnosed with a malignant tumor based on a first TGS. If the TGS indicates that the patient is high risk or intermediate risk, a therapeutic agent that increases the number of CD137+ tumor-infiltrating leukocytes is administered.

The therapeutic may be an immunomodulator, such as a chemoattractant, an antibody, a cytokine, a chemokine, a small molecule, or a kinase inhibitor. One example of a chemoattractant is chemerin (RARRES2 [retinoic acid receptor responder (tazarotene induced) 2]). Chemerin is a chemoattractant polypeptide that attracts cells including NK cells, T cells, macrophages and dendritic cell subsets. Chemerin is down-regulated in a variety of tumors including, e.g., melanoma, adenocarcinomas, adenomas, and carcinomas.

In one embodiment, administering the therapeutic lowers the first TGS of the patient, thereby providing a second TGS. The reduction in TGS may place the patient in a different risk category. For example, the first TGS indicates the patient is high risk and the second TGS indicates the patient is intermediate risk or low risk. In another example, the first TGS indicates the patient is intermediate risk and the second TGS indicates the patient is low risk. As described in Example 2, an International Prognostic Index (IPI) score may be combined with the first TGS to indicate risk.

Following administration of the therapeutic, one or more additional therapeutic agents can optionally be administered to the patient. For example, the patient may then be treated with the current standard of care, rituximab, cyclophosphamide, doxorubicin, vincristine, and prednisone (R-CHOP). Alternatively, the therapeutic may be administered in combination with or simultaneously with one or more other therapeutic agents.

In one embodiment, the tumor-infiltrating leukocytes express CD137, and in particular, the tumor-infiltrating leukocytes are NK cells or T cells.

In another embodiment, a method of treating a patient diagnosed with a malignant tumor and having a first two gene score (TGS), wherein the first TGS indicates the patient is high risk, intermediate risk, or low risk, comprises administering a therapeutic that lowers the TGS of the tumor. In particular, the therapeutic leads to increased expression of TNFRSF9 in the tumor, thereby decreasing the TGS.

It is contemplated that the methods described herein will increase the number of tumor-infiltrating leukocytes and/or decrease the TGS of the tumor, and thereby increase the likelihood that the patient will be responsive to a standard treatment protocol, such as CHOP or R-CHOP.

Administration of a therapeutic agent, in pure form or in an appropriate pharmaceutical composition, can be carried out via any of the accepted modes of administration of agents for serving similar utilities. The pharmaceutical compositions can be prepared by combining a therapeutic agent with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition.

Typical routes of administering therapeutics and related pharmaceutical compositions include, without limitation, oral, topical, transdermal, inhalation, parenteral, sublingual, buccal, rectal, vaginal, intranasal, and intratumor. The term parenteral as used herein includes subcutaneous injections, intravenous, intramuscular, intrasternal injection or infusion techniques. Preferred modes of administration depend upon the nature of the therapeutic. For example, the therapeutic may be administered locally at the tumor site (e.g., intratumor injection), or the therapeutic may be administered systemically. An amount that, following administration, leads to an increase in the number of tumor-infiltrating leukocytes is considered effective.

The therapeutic may be administered alone or in combination with other known cancer treatments, such as radiation therapy, chemotherapy, transplantation, immunotherapy, hormone therapy, photodynamic therapy, etc. Such additional therapeutic agents may be accepted in the art as a standard treatment for a particular disease state as described herein (e.g., R-CHOP), such as a tumor, cancer or a proliferative disease or disorder. Exemplary therapeutic agents contemplated include cytokines, steroids, chemotherapeutics, radiotherapeutics, or other active and ancillary agents.

In a particular embodiment, the therapeutic is administered in combination with CHOP or R-CHOP. In another embodiment, the treatment is sequential, wherein the therapeutic is administered first, and after a period of time, CHOP or RCHOP is administered to the patient.

### EXAMPLES

The following examples are intended to illustrate but not limit the disclosure.

### Example 1

### Two Gene Score Predicts Overall Survival

The TGS diagnostic test was developed in order to more accurately assess patient risk in DLBCL (Alizadeh et al. Blood 118:1350-1358, 2011). The TGS evaluates both the tumor and the tumor environment to determine a patient's overall clinical risk, e.g., overall survival, response to treatment, and chance of relapse.

TNFRSF9 was the best univariate and bivariate predictor among genes more highly expressed in non-tumor cells. Bivariate combinations of genes with LMO2 were tested for their ability to predict survival using multivariate Cox regression, limited to genes with higher expression among non-tumor cells (CD19−) as compared with paired t-test (p<0.05) to matched tumors cells (CD19+), and exhibiting significant (p<0.01) univariate predictive value in R-CHOP treated patients (DLBCL1). Within this group of genes, TNFRSF9 was identified as the best univariate predictor and also bivariate partner for LMO2. The top 15 genes are shown below in Table 1 and are ranked by p-value of the bivariate model.

TABLE 1

| | | | | | Tumor |
|---|---|---|---|---|---|
| | | | | | vs. |
| | | Association with overall survival | | | Micro- |
| | | In RCHOP training cohort | | | Environment |
| | | (DLBCL1) | | | (CD19+ vs |
| Entrez | | | | Bivariate | CD19−) |
| Gene | Gene | Univariate | | (with LMO2) | p-value |
| Identifier | Symbol | z-score | p-value | p-value | (paired T-test) |
| 3604 | TNFRSF9 | −4.30 | 1.72E−05 | 2.33E−08 | 0.04 |
| | (CD137) | | | | |
| 5578 | PRKCA | −3.21 | 1.32E−03 | 1.04E−07 | 0.01 |
| 5334 | PLCL1 | −3.22 | 1.29E−03 | 1.67E−07 | 0.01 |
| 64375 | IKZF4 | −3.71 | 2.09E−04 | 1.93E−07 | 0.04 |
| 80273 | GRPELI | 3.23 | 1.24E−03 | 1.06E−06 | 0.00 |
| 81615 | TMEM163 | −3.09 | 2.03E−03 | 1.17E−06 | 0.02 |
| 56548 | CHST7 | 3.80 | 1.44E−04 | 1.46E−06 | 0.04 |
| 113763 | C7orf29 | −3.15 | 1.61E−03 | 1.65E−06 | 0.00 |
| 321 | APBA2 | −3.05 | 2.26E−03 | 1.74E−06 | 0.05 |
| 3687 | ITGAX | −3.70 | 2.20E−04 | 2.33E−06 | 0.01 |
| 286133 | SCARAS | −2.60 | 9.36E−03 | 3.65E−06 | 0.05 |
| 5919 | RARRES2 | −2.74 | 6.07E−03 | 4.58E−06 | 0.01 |
| 2060 | EPS15 | −3.22 | 1.29E−03 | 5.04E−06 | 0.03 |
| 6355 | CCL8 | 2.76 | 5.74E−03 | 6.25E−06 | 0.01 |
| 4208 | MEF2C | −2.75 | 5.92E−03 | 1.45E−05 | 0.01 |

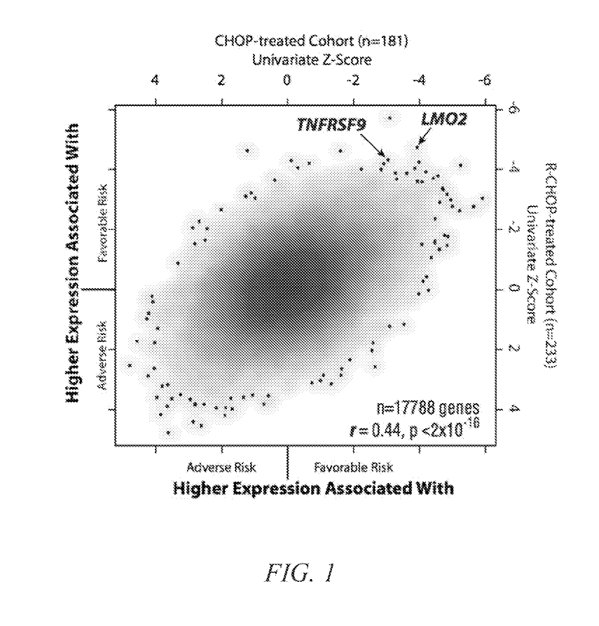

The univariate associations between genome-wide expression, survival, and therapeutic era (R-CHOP vs. CHOP) were also evaluated. Expression profiles of 17,888 genes were tested for their association with overall survival in 414 patients treated with R-CHOP (DLBCL1, y-axis) or CHOP (DLBCL2, x-axis) as shown in FIG. 1. Each point depicts a single gene, and saturation of color within the shaded central cloud represents gene density (frequency of genes within a region); single genes occurring in low density regions are indicated by a black point. LMO2 was among the best univariate predictors considering all measured genes, in both R-CHOP and CHOP treated patients. The position of TNFRSF9 is also indicated. Genes generally tend to have significant positive correlation in prognostic influence when comparing therapeutic eras (Pearson r=0.44, p<2×10-16).

In order to measure expression of the two genes (LMO2 and TNFRSF9) involved in the TGS from tumor samples, RNA was extracted from two 5-um-thick slices of FFPE tissue sections, as long as visual inspection finds at least 3 mm of embedded tissue within the block. Quality and yield assessments were made for RNA and cDNA by optical spectra assessing the OD260/280 ratio and size distribution. A mix of unlabeled PCR primers and TaqMan minor groove binder probe (FAM dye-labeled) were used for measurement of expression of LMO2 (Hs00277106_m1) and tumor necrosis factor receptor superfamily member 9 (TNFRSF9; Hs00155512_m1). Phosphoglycerate kinase 1 (PGK1) with VIC-dye labeled Pre-Developed Assay Reagent (Applied Biosystems) was used for an endogenous control. For calibration, cDNA from the Raji lymphoma cell line (ATCC) was run in parallel, obtaining delta-delta-Ct values for TNFRSF9 and LMO2 in each sample. Real-time RT-PCR was then performed to obtain expression values for LMO2 and TNFRSF9.

The two gene score (TGS) was then calculated using the following equation:

TGS=(−0.32×LMO2)+(−0.29×TNFRSF9).

### Example 2

### Composite TGS-IPI Score

In order to obtain a composite model (i.e., the TGS-IPI) incorporating LMO2, TNFRSF9, and IPI, a multivariate Cox regression was applied to the training set, with the independent variables being the previously computed TGS, together with the traditional IPI score of each patient (on a scale ranging from 0 to 5) (Alizadeh et al. Blood 118:1350-1358, 2011). A constant (4) was added as a term within the TGS-IPI to avoid negative scores, based on the lowest non-adjusted measurement observed across studies. As with the TGS, the TGS-IPI was computed in the test sets using the same parameters derived in the training cohort (DLBCL1). The following equation was used to calculate TGS-IPI:

TGS-IPI=(0.93×TGS)+(0.6×IPI)+4.

Alternate versions of the TGS with equal weightings of LMO2 and TNFRSF9 (i.e., TGS=−LMO2−TNFRSF9) and the TGS-IPI (TGS-IPI=2×IPI−LMO2−TNFRSF9) yield nearly identical results within test and validation cohorts and can be used to determine TGS and TGS-IPI.

### Example 3

### TNFRSF9 Expression and Patient Stratification

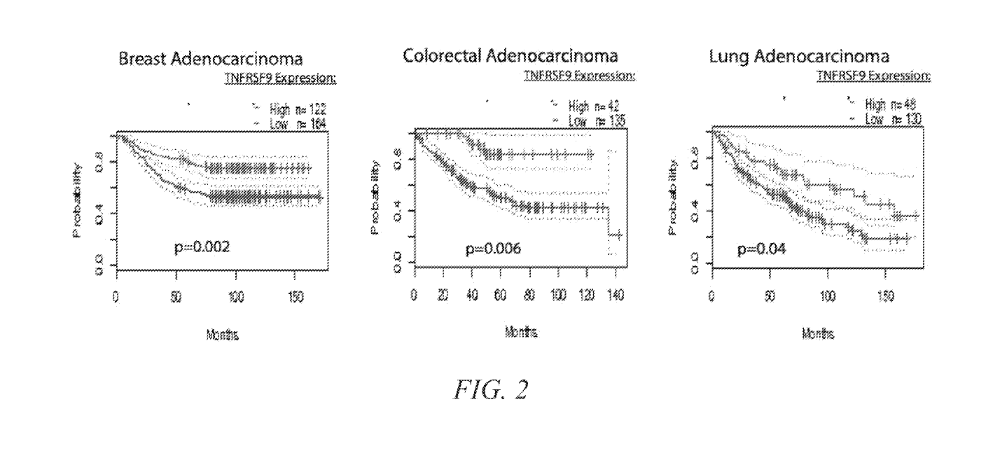

The stratification of patient outcomes based on level of expression of TNFRSF9 in patients with adenocarcinomas of the breast (left), colon (middle), and lung (right) is shown in FIG. 2. The left panel depicts distant metastasis free survival (DMFS) of a cohort of 286 women previously described (NKI/Rotterdam, GSE2034) as diagnosed with early stage breast adenocarcinomas between 1980-1995 and without lymph node involvement at presentation (Age 26-83, ER+73%, PR+58%). The middle and right panels depict overall survival (OS) for cohorts of patients as diagnosed with colorectal adenocarcinomas (n=177, GSE17536) and lung adenocarcinomas (n=179 [University of Michigan], NCI-caArray: jacob-00182-UM), respectively. Depicted strata reflect patients classified as expressing High (upper curve), or Low (lower curve) levels of TNFRSF9 mRNA as measured on HG-U133 series Affymetrix microarrays, with dotted lines representing 95% confidence intervals above and below each curve.

High and Low labels related to an idealized cut-point of TNFRSF9 expression exhibiting strongest association with the depicted outcome, and meriting consideration after correction for multiple hypothesis testing. After such correction, the two-sided log-rank p-values the dichotomous stratifications depicted in the left, middle, and right panels were p=0.005, 0.004, and 0.06, respectively. The corresponding estimates for the association between continuous expression of TNFRSF9 and as measured by the log-likelihood test within a univariate Cox regression model was p=0.002, 0.006, and 0.04, respectively. This data shows that TNFRSF9 can be used in other types of tumors as well as lymphoma to predict patient outcomes.

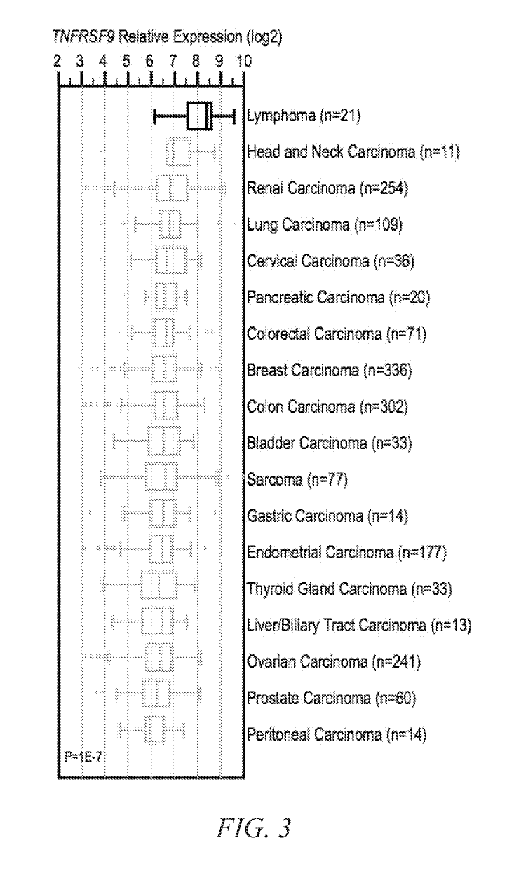

Furthermore, FIG. 3 shows the measured expression of TNFRSF9 in several human tumor types.

### Example 4

### TGS and TGS-IPI Patient Stratification

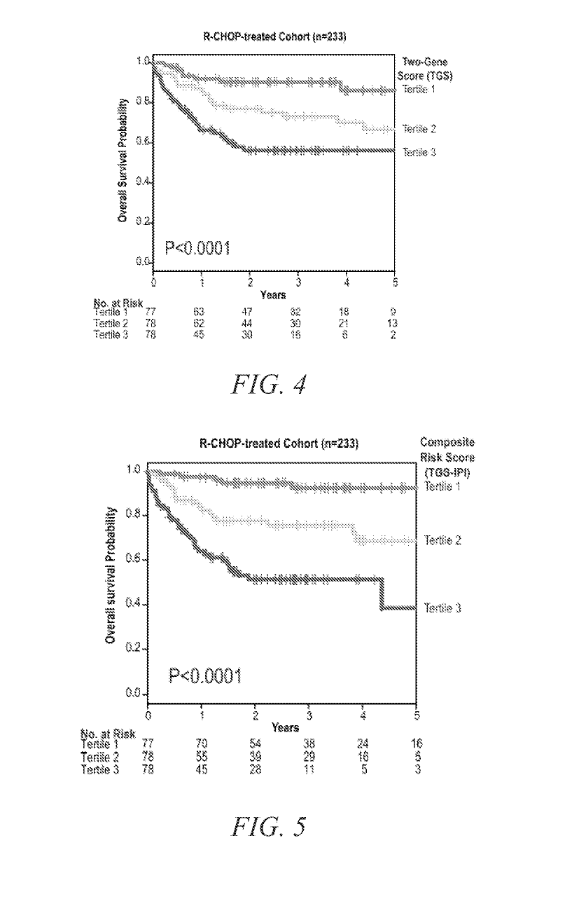

In order to demonstrate the performance of the TGS, the expression of LMO2 and TNFRSF9 as evaluated in the training (DLBCL1) cohort described above was stratified. As shown in FIG. 4, tertiles of the TGS define three risk groups with distinct Kaplan-Meier estimates of survival (high risk, TGS>−0.91; intermediate risk, −0.91>=TGS>−1.60; and low risk, TGS<=−1.60). Similarly, FIG. 5 shows Kaplan-Meier estimates of strata using tertiles of a composite risk score integrating TGS and IPI (TGS-IPI). Depicted p values reflect log-likelihood estimates.

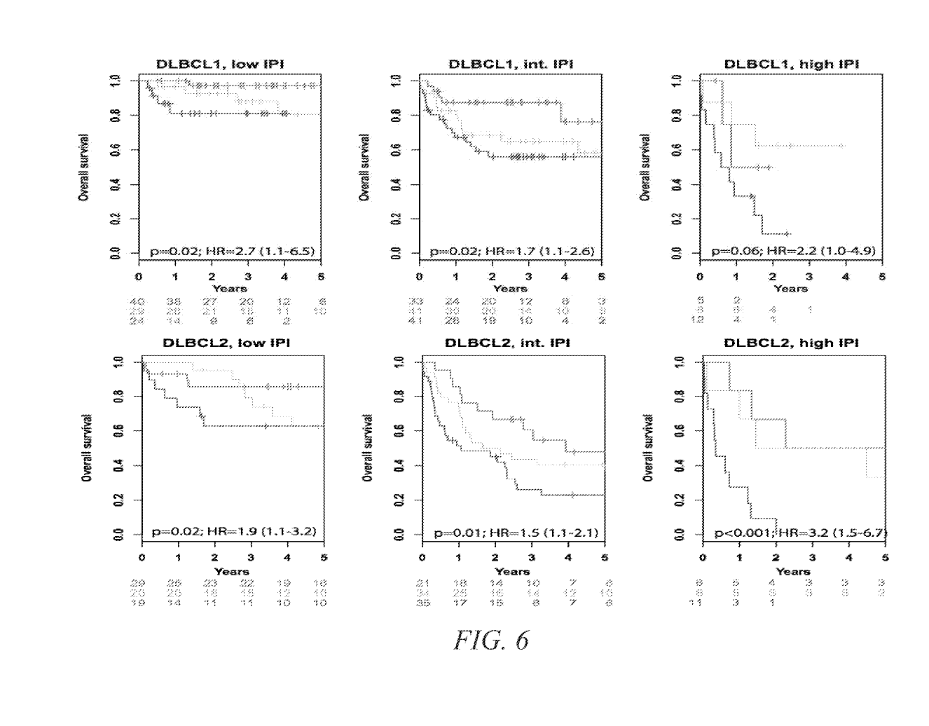

FIG. 6 shows Kaplan Meier analyses of TGS risk groups can further stratify individual IPI risk categories of patients treated with RCHOP (top; DLBCL1), or CHOP (bottom; DLBCL2), including patients with (left panels) Low (IPI=0,1), (middle panels) Intermediate (IPI=2,3), or (right panels) High IPI Risk (IPI=4,5). The data shows that the TGS was able to further risk stratify patients independent of the IPI and added prognostic value.

### Example 5

### Tumor-Infiltrating Leukocytes

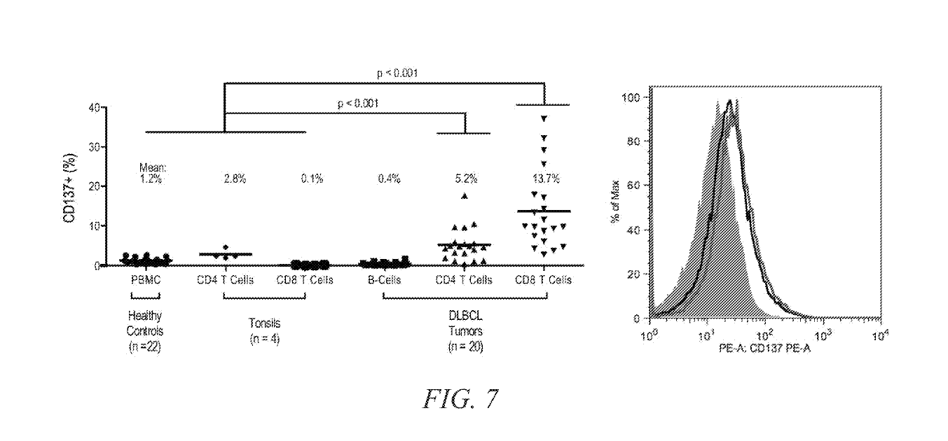

TNFRSF9 expression is associated with improved clinical outcomes, as higher expression levels correlate with less risk, and a lower TGS. In order to examine the expression of TNFRSF9 in tumor-infiltrating leukocytes, cells were examined using flow cytometry. FIG. 7 shows the expression of CD137 (TNFRSF9) on T cells (left) and NK cells (right) present within DLBCL biopsy samples.

A mouse model of melanoma was used to study the effect of an immunomodulator, chemerin (RARRES2) (Pachynski et al. J. Exp. Med. 209(8):1427-1435, 2012). Both control and chemerin-over expressing tumors were transplanted into mice. Intratumoral injections of chemerin were also evaluated. For all animal experiments, C57BL/6 or B6.129S7-Rag1tm1Mom/J (RAG1 KO) mice were purchased from the Jackson Laboratory. Murine B16F0 melanoma, L1.2 B lymphoma, and human 293 HEK lines were obtained from the American Type Culture Collection. Cell lines were grown in complete media consisting of RPMI 1640 supplemented with 10% FBS, sodium pyruvate, penicillin/streptomycin, and beta-mercaptoethanol.

To evaluate the effect of constitutively secreted chemerin on tumor growth, control or chemerin-expressing B16 melanoma tumor cells (0.5×10⁶ or 1×10⁶) were inoculated subcutaneously into mice. Tumor growth was measured every 2-4 d by calipers, and size was expressed as the product of perpendicular length by width in square millimeters. Mice were euthanized when tumor size reached about 400 mm² or when tumor sites ulcerated or at day 17 to evaluate for tumor-infiltrating leukocytes (TILs) by FACS.

To evaluate the effect of exogenous chemerin on tumor growth, tumor lines were inoculated as indicated. Recombinant, active, carrier-free murine chemerin (R&D Systems) was reconstituted in sterile PBS. Endotoxin levels were <1.0 EU per 1 μg of the protein by the limulus amebocyte lysate method, as reported by the manufacturer. Purified murine serum albumin (Sigma-Aldrich) or PBS was used as control as indicated. 50-100 μl chemerin or control was injected both inside and around the periphery of the tumor mass. Tumors were injected once daily as indicated with either control or chemerin. Tumors were either treated from time of inoculation or after establishment at approximately day 5-10, when tumors were palpable.

For experiments using established tumors, mice were inoculated and randomly divided into two groups before treatment. For depletion experiments, wild-type mice were treated with anti-Asialo GM1 polyclonal antiserum (Wako Chemicals USA) to deplete NK cells. Anti-Asialo GM1 was reconstituted in water per the manufacturer's recommendations, diluted 1:10 in PBS, and 200 μl was injected i.p. the day before tumor inoculation. 100 μl was then injected i.p. on day 0, and every 3-4 d thereafter for the duration of the experiment. Sterile PBS was injected i.p. in an identical fashion in control groups. To ensure NK depletion, 50-100 ml of peripheral blood from mice undergoing NK cell depletion with anti-Asialo GM1 was collected by retroorbital bleed under isoflurane anesthesia at days 3-5 after first injection with anti-Asialo GM1. Red blood cell lysis was performed using ACK lysis buffer (Sigma-Aldrich). Samples were then stained for flow cytometry using anti-CD3 and either anti-NK1.1 or anti-DX5 monoclonal antibodies).

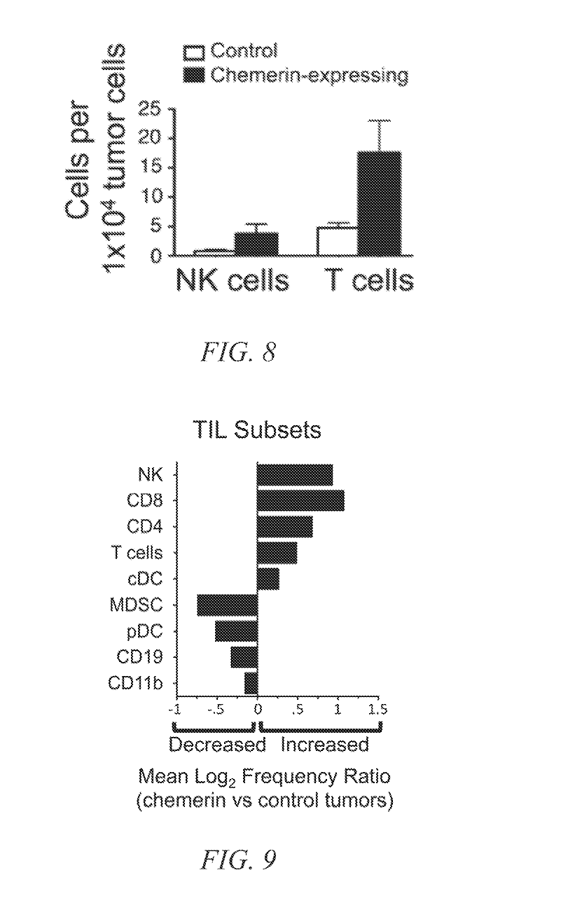

FIG. 8 shows the increase in NK and T cells in murine tumors treated with chemerin. FIG. 9 shows the effect of chemerin on infiltrating leukocyte subsets in murine tumors. The immunomodulator increased the representation of activated NK and T cells within the tumor.

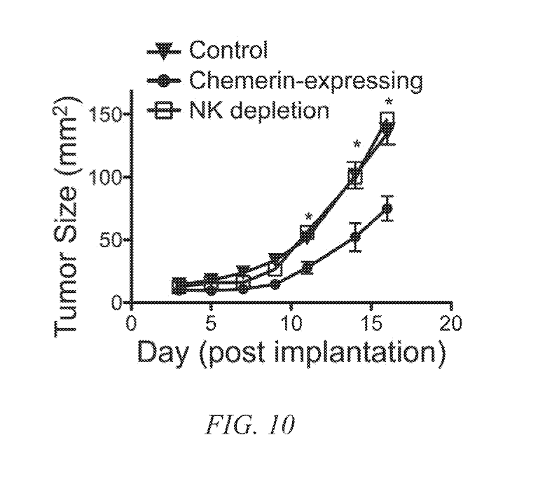

FIG. 10 shows the clinical effect of chemerin on tumor growth in mice. Depletion of NK cells within the tumor abrogated the clinical efficacy of the immunomodulatory peptide.

This example shows that an immunomodulator, such as chemerin, can be used to increase the number of tumor-infiltrating leukocytes, particularly CD137+NK cells, and thereby inhibit tumor growth.

### Example 6

### Two Gene Score Animal Model

The TGS may be modeled in a non-human animal. In this example, mice are utilized to model the TGS. In order to establish the model, a number of mice are inoculated (e.g., subcutaneously or intravenously) with tumor cells that have been grown in vitro in culture, or mice with spontaneous or inducible tumors are used. The tumor may be of varying species origin (e.g., murine or human).

Tumor cells are allowed to grow in the mice until tumor nodules are palpable or detectable. Once the tumors are detectable, mice are anesthetized and a portion of the tumor nodule(s) is/are resected, placed in a freezing compound, and frozen or fixed and embedded in paraffin. The mice are allowed to recover from the survival surgery.

A portion of the tumor is sectioned and RNA is obtained. The RNA is utilized in RT-PCR as described in Example 1, to obtain values for LMO2 and TNFRSF9. The TGS is calculated as described above.

The survival data of the mice are recorded. The clinical outcomes of the mice are then correlated with their calculated TGS scores. Risk groups are established as described above in Example 4.

### Example 7

### Screening Therapeutics Using the TGS Animal Model

The TGS animal model may be used in a therapeutic setting in order to evaluate a therapeutic. A number of mice with tumors of a certain TGS risk group (e.g., high, intermediate, or low) are identified. The mice are then treated with a therapeutic agent or a control/placebo agent. The TGS is re-evaluated, as above.

Response rates and clinical outcomes of the mice are assessed. The TGS from prior to treatment and after treatment are compared. The change in TGS is correlated with the response rates and clinical outcomes of the mice. Change in TGS (pre- and post-treatment), response rates, clinical outcomes, and correlations are compared between groups to determine the effect of the therapeutic.

### Example 8

### Evaluating Patient Response with TGS

TGS may be used in humans with malignancies to evaluate a response to therapy or a particular therapeutic. The TGS is calculated for a patient, as described above. The TGS is utilized to classify the patient into a risk category (e.g., high, intermediate, or low). The patient undergoes a treatment or therapy intended to treat the malignancy or a disease state.

After a certain length of treatment, the patient undergoes a repeated evaluation of the TGS for their malignancy. The pre- and post-treatment TGS scores are compared. Response and clinical outcome are assessed. Additional histologic or other evaluation of the tumor is then correlated with the TGS.

### Example 9

### Treating Cancer in a Patient with a Calculated TGS

The treatment of a cancer patient is based on the TGS. In this example, the TGS is high, indicating a relative low expression level of TNFRSF9 in the tumor biopsy. The patient is selected because of the high TGS, and a treatment or therapy may be more likely to benefit this patient with a poor risk as determined by the TGS.

### Example 10

### TGS Mouse Model with Chemerin-Secreting Tumors

In order to determine the effect of chemerin on the TGS components in a mouse model, B16 melanoma tumors were implanted subcutaneously in C57BL6 mice. One cohort of mice received control tumors that received no treatment. The other cohort received tumors that were engineered to secrete the chemoattractant chemerin locally, thereby mimicking intratumoral injections of the chemoattractant. Tumor expression of chemerin resulted in an increase in tumor-infiltrating leukocytes, more specifically NK and T cells (FIG. 9).

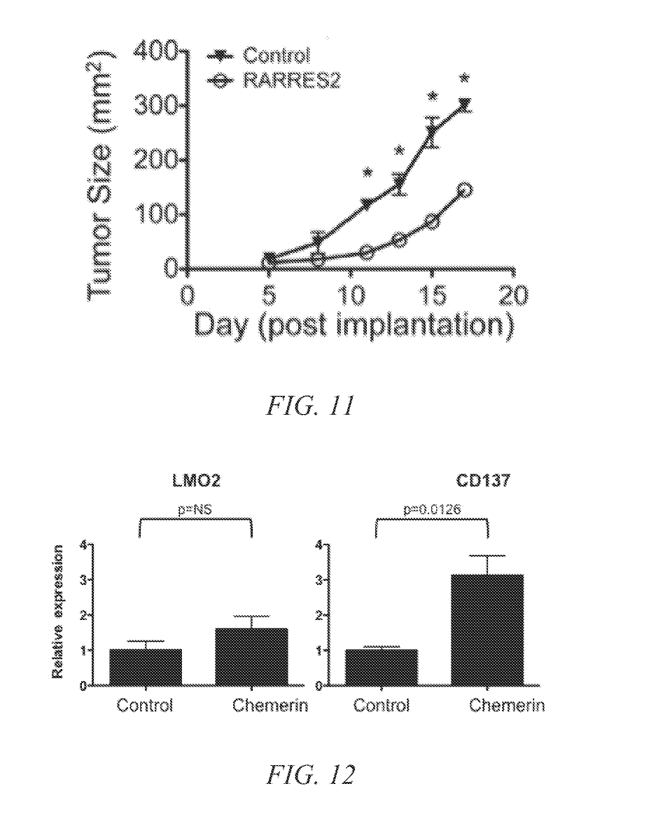

Clinically, the changes in the number of tumor-infiltrating leukocytes also correlated with tumor growth as well. As shown in FIG. 11, chemerin (RARRES2) secreting tumors were significantly smaller than control tumors in tumor-bearing C57BL6 mice (*p<0.05), which is an indicator of improved clinical outcome.

After a period of 17 days, tumors were analyzed by RT-qPCR for LMO2 and CD137 (TNFRSF9), the key components of the TGS. Analysis of the tumors showed that the treatment of the tumors with the chemoattractant chemerin resulted in a significantly increased CD137 (TNFRSF9) expression in the chemerin tumors (n=5) compared to the control group (n=4) that did not receive treatment. Importantly, the expression level of LMO2 in both cohorts was not significantly changed (FIG. 12). Accordingly, a calculated TGS from the tumors would result in improved/lowered TGS score for the chemerin-treated group, whereas the control group that was not treated, and thus had fewer infiltrating NK and T cells, would have a worse/higher TGS score. GAPDH was used as the housekeeping/reference gene (not shown).

### Example 11

### TGS Mouse Model with Chemerin-Secreting and EGFR-Expressing Tumors

In a separate experiment, three cohorts of mice were inoculated with B16 melanoma tumors. One group had control tumors, one group had tumors that expressed human EGFR, and the third group had tumors secreting the chemoattractant chemerin, mimicking intratumoral injection or treatment with chemerin. Each group comprised three mice. Tumors were allowed to grow until Day 9. On Day 9, both control and EGFR-expressing tumor groups received treatment with the therapeutic anti-human EGFR antibody, cetuximab (200 ug injection intraperitoneally per mouse). Forty-eight hours after the single injection (Day 11), the tumors from all three groups were analyzed for infiltrating NK and T cells, as well as CD137 (TNFRSF9) expression.

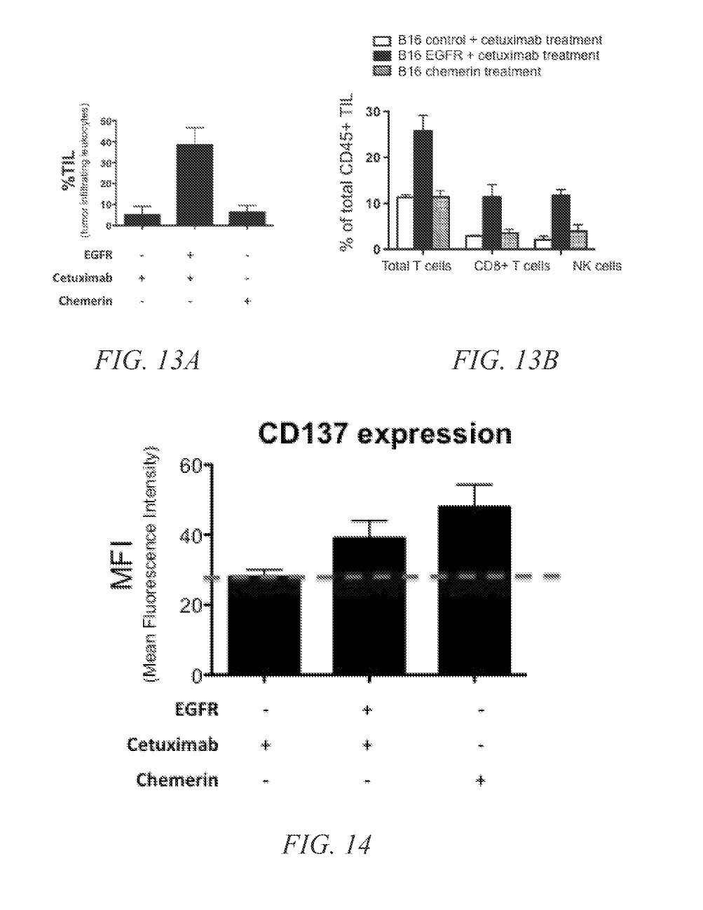

FIG. 13A shows the increase in total tumor-infiltrating leukocytes (TIL) that was observed in the cohort of mice bearing B16 melanoma tumors that express EGFR and were treated with the therapeutic cetuximab. The group bearing the B16 melanoma tumors that did not harbor the EGFR tumor antigen, and thus could represent a population of non-responders (e.g., cetuximab-resistant) or those that do not have specific tumor antigens, but that did get treated with cetuximab showed a significantly lower percentage of tumor-infiltrating leukocytes (TIL). FIG. 13B shows levels of both NK and T cells for the three cohorts. The chemerin-treated tumors showed small increases in both CD8+ T cells and NK cells.

FIG. 14 shows the measured CD137 expression on total tumor-infiltrating leukocytes in the three cohorts. EGFR-expressing tumors (representing a “responder” cohort) and those treated with chemerin showed an increase in CD137 expression by flow cytometric analysis (FACS) compared to control tumors treated with cetuximab, representing a “non-responder” cohort.

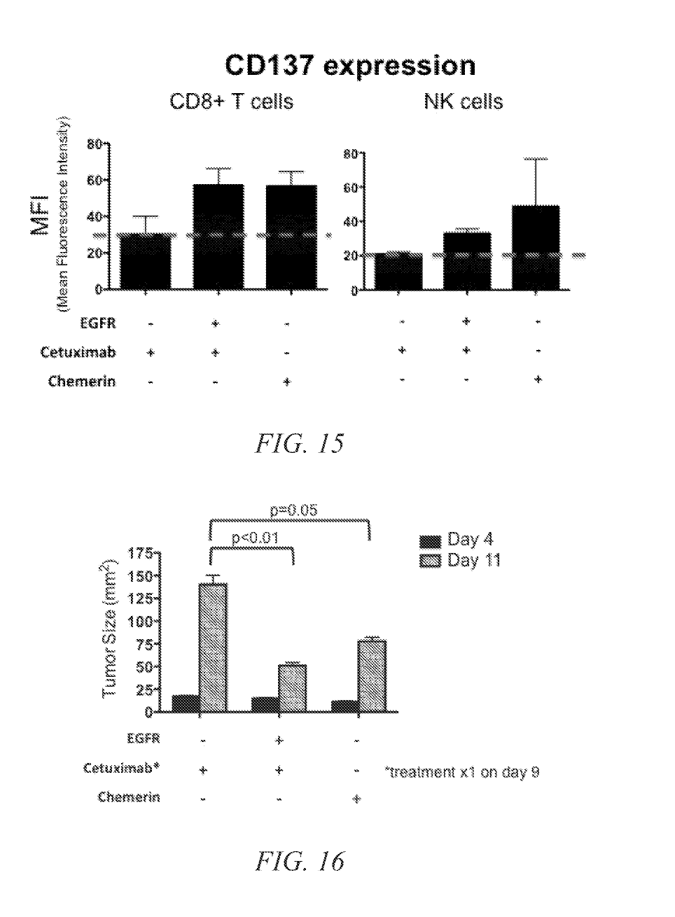

Further analysis of the tumor-infiltrating leukocytes by FACS showed similar increases in CD137 expression on tumor-infiltrating NK and T cells in both the EGFR-expressing and chemerin-secreting B16 melanomas compared to the cetuximab-treated control tumors (FIG. 15).

The same tumors from the three groups were analyzed for tumor size (mm²), a surrogate for clinical outcomes and survival, at pretreatment (Day 4) and post-treatment (Day 11). EGFR-expressing tumors treated with cetuximab and chemerin-secreting tumors were both significantly smaller than control tumors (not expressing EGFR) treated with cetuximab (FIG. 16). These results indicated that, in addition to the resultant increase in CD137 expression as above, tumor growth was significantly slowed in both the EGFR-expressing and chemerin-treated groups.

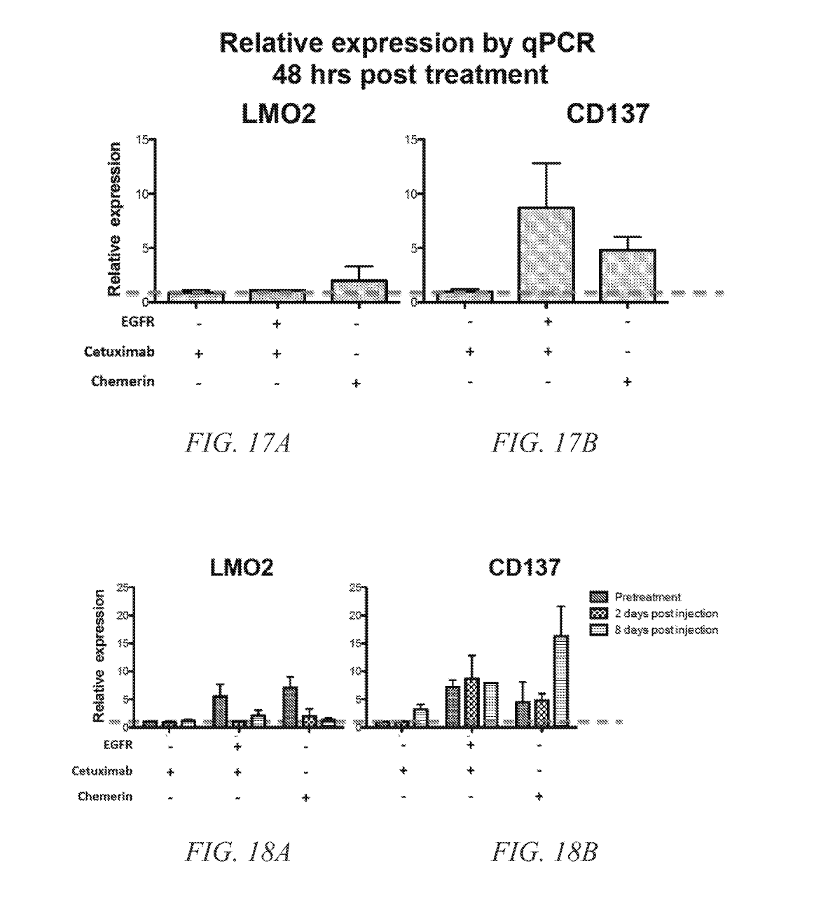

Tumor biopsies from the three treatment groups were also analyzed by RT-qPCR to measure LMO2 and CD137 expression. As shown in FIG. 17A, at 48 hours post treatment (Day 9) the expression level of LMO2 was similar between the three groups. However, at the same time point, CD137 expression was significantly higher in both the tumors treated with chemerin and the EGFR-expressing tumors treated with cetuximab in comparison to the EGFR-expressing tumors that did not receive cetuximab treatment (FIG. 17B). FIGS. 18A and 18B show the relative expression levels of LMO2 and CD137, respectively, pretreatment (Day 4), 2 days post treatment (Day 11), and 8 days post treatment (Day 17).

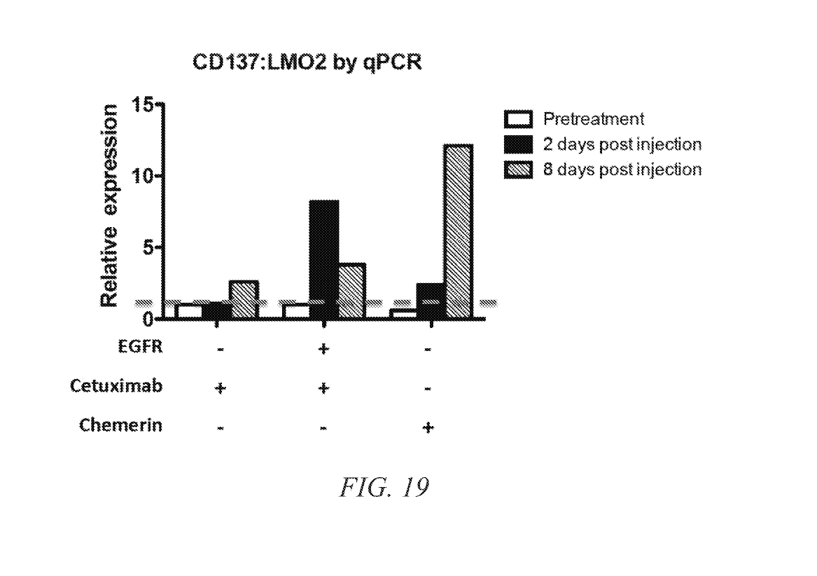

The TGS takes into account the relative expression levels of both CD137 and LMO2. Therefore, the ratio of relative expression of CD137 to LMO2 was determined. As shown in FIG. 19, the CD137:LMO2 ratio was greater in both the chemerin treated and EGFR-expressing and cetuximab treated tumors in comparison to the non-EGFR and cetuximab treated tumors.

The data provided herein demonstrate that an immunomodulatory therapeutic, such as an antibody (e.g., cetuximab) or a novel chemoattractant (e.g., chemerin), can favorably alter the immune infiltrate in treated tumors, resulting in an increase in NK and T cells in the tumor. The data show that after treatment, the expression level of CD137 increased on these cells (as shown by FACS) and in the tumor as a whole (as shown by RT-qPCR). These results demonstrate that the level of LMO2 by RT-qPCR does not significantly change between control and chemerin-treated tumors while the expression of CD137 is significantly increased, therefore, the calculated TGS would be lowered. The decrease in TGS corresponds with a decrease in the observed tumor size in vivo.

### Example 12

### TGS Mouse Model with Cetuximab Treatment of EGFR-Expressing Tumors

In order to further evaluate the effect of cetuximab treatment on CD137 expression, two additional tumor types, head and neck squamous cell carcinoma (SCC6 cells) and breast adenocarcinoma (TUBO cells), were used. The tumor cells used in this example were genetically modified to express EGFR.

### Head and Neck Squamous Cell Carcinoma

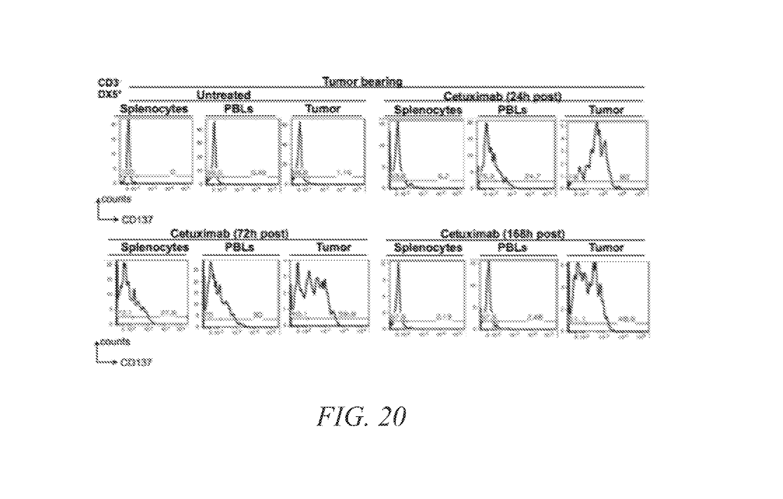

Nu/nu mice were inoculated with 1×10⁶ EGFR-expressing SCC6 tumor cells subcutaneously on the left flank, and cetuximab was administered on Day 21 post-tumor inoculation. FIG. 20 shows representative CD137 expression on CD3-DX5+NK cells isolated from the spleen, peripheral blood, and intratumoral that were analyzed for CD137 expression 24, 72, or 168 hours following cetuximab treatment or isotype control in tumor-bearing mice (n=3 mice per group).

### Breast Adenocarcinoma

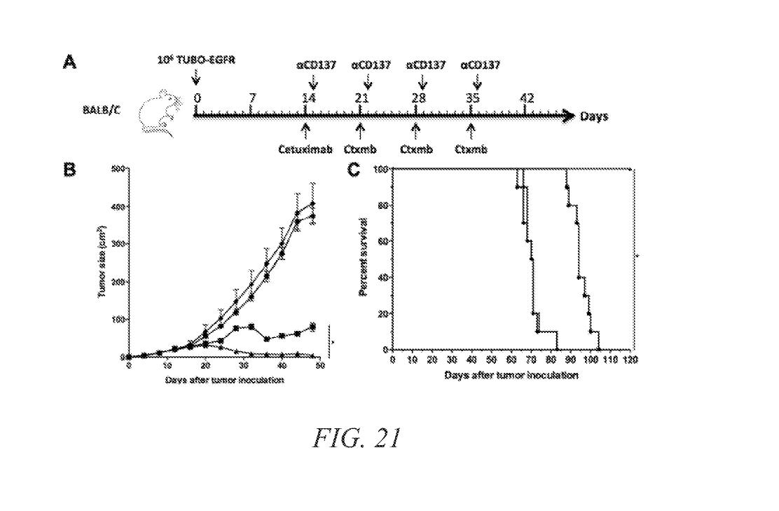

In another experiment, using EGFR-expressing TUBO breast adenocarcinoma tumors, mice were treated with both cetuximab and an anti-CD137 agonistic antibody to look at the impact of treatment on tumor size and survival. BALB/c mice were inoculated with 1×10⁶ TUBO-EGFR tumor cells subcutaneously on the left flank, and cetuximab and/or anti-CD137 mAb was administered starting day 14 post-tumor inoculation (FIG. 21A). Post-tumor inoculation, mice then received either Rat IgG control on Day 14 (), cetuximab on Day 14 (▪), anti-CD137 antibody on Day 15 (♦), or cetuximab on Day 14 and anti-CD137 antibody on Day 15 (▴) with each injection repeated weekly for 4 total injections. Mice (10 per group) were then monitored for tumor growth (FIG. 21B) and overall survival (FIG. 21C).

As shown herein, identical treatment of both non-responder (e.g., non-EGFR bearing) and responder (e.g., EGFR-expressing) cohorts with an antibody (e.g., cetuximab) resulted in differences in tumor-infiltrating NK and T cells. Furthermore, this corresponded to differences in CD137 expression levels in the tumor. Specifically, responding populations showed an increase in NK and T cells in the tumors along with corresponding increases in CD137 expression. This finding along with the in vivo survival data demonstrates that treatment with immunomodulatory agents results in changes in tumor-infiltrating immune cells, that increases in CD137 expression can be monitored by RT-qPCR and TGS, and that these changes correlate with clinical outcomes.

### Example 13

### In Vitro TGS Model

In order to determine if the two gene score (TGS) can be evaluated in vitro, a co-culture assay system that utilizes the main components from the tumor microenvironment, namely tumor cells and immune cells, was developed. EGFR-expressing B16 melanoma cells were cultured with whole splenocytes isolated from C57BL6 mice. The test group received cetuximab (anti-EGFR antibody), while the control group did not receive cetuximab. The rational for the experimental design was that the antibody enables and activates NK cells and other cells involved in antibody-dependent cell-mediated cytotoxicity (ADCC) to mediate killing of the tumor cells. The activated immune cells express CD137.

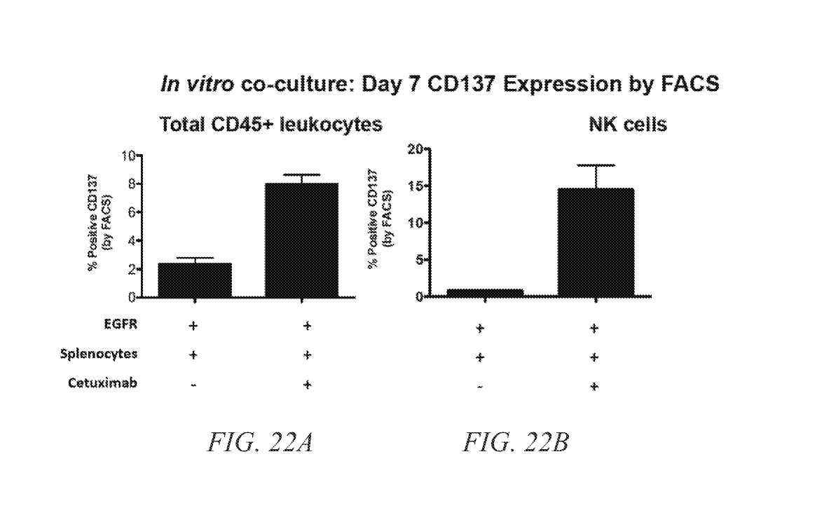

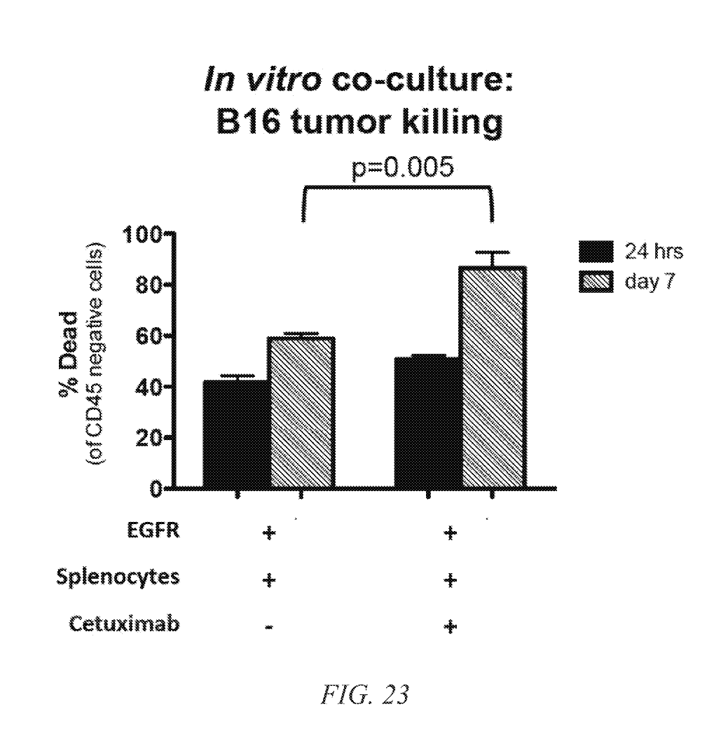

At Day 7 of in vitro co-culture, the percentage of both CD45+ leukocytes (FIG. 22A) and NK cells (FIG. 22B) that were CD137+ significantly increased in cultures that received cetuximab in comparison to the control cells. Additionally, in order to determine the percentage of B16 tumor cells that were killed in the co-culture environment with and without added cetuximab, the percentages of CD45-cells, representing the non-leukocyte tumor cells in the co-culture, that were dead were determined at 24 hours and Day 7 of the in vitro co-culture. As shown in FIG. 23, there was a significantly greater amount of dead CD45-cells in cultures that received cetuximab.

These results demonstrate that therapeutic agents can be evaluated in vitro to examine the effect on TGS. In particular, an increase in killing of tumor cells in the treated group and a corresponding increase in CD137 expressing leukocytes and NK cells were observed.

All patents, patent applications, and references cited herein are incorporated in their entireties by reference. Unless defined otherwise, technical and scientific terms used herein have the same meaning as that commonly understood by one of skill in the art.

What is claimed is:

1. A method of treating a patient diagnosed with a malignant tumor and having a first two gene score (TGS), wherein the first TGS indicates the patient is high risk, intermediate risk, or low risk, the method comprising administering a therapeutic that leads to an increased number of tumor-infiltrating leukocytes.

2. The method of claim 1, wherein the malignant tumor is a lymphoma or an adenocarcinoma.

3. The method of claim 2, wherein the lymphoma is a non-Hodgkin lymphoma.

4. The method of claim 3, wherein the non-Hodgkin lymphoma is diffuse large B-cell lymphoma (DLBCL).

5. The method of claim 2, wherein the adenocarcinoma is a breast adenocarcinoma, a colon adenocarcinoma, or a lung adenocarcinoma.

6. The method of claim 1, wherein the therapeutic is an immunomodulator.

7. The method of claim 6, wherein the immunomodulator is a chemoattractant, an antibody, a cytokine, a chemokine, a small molecule or a kinase inhibitor.

8. The method of claim 7, wherein the chemoattractant is chemerin.

9. The method of claim 1, wherein the therapeutic is administered locally.

10. The method of claim 9, wherein the therapeutic is administered via intratumor injection.

11. The method of claim 1, wherein the administering lowers the first TGS of the patient, thereby providing a second TGS that is lower than the first.

12. The method of claim 11, wherein the first TGS indicates the patient is high risk and the second TGS indicates the patient is intermediate risk or low risk.

13. The method of claim 11, wherein the first TGS indicates the patient is intermediate risk and the second TGS indicates the patient is low risk.

14. The method of claim 1, further comprising administering one or more chemotherapeutic agents to the patient.

15. The method of claim 14, wherein the chemotherapeutic agents comprise rituximab, cyclophosphamide, doxorubicin, vincristine, and prednisone (R-CHOP).

16. The method of claim 1, wherein an International Prognostic Index (IPI) score is combined with the first TGS to indicate risk.

17. The method of claim 1, wherein the tumor-infiltrating leukocytes express CD137.

18. The method of claim 17, wherein the tumor-infiltrating leukocytes are NK cells or T cells.

19. A method of modeling TGS in a non-human animal comprising:
(a) obtaining a first sample from a tumor in a non-human animal,
(b) determining a TGS,
(c) observing a clinical outcome of the non-human animal, and
(d) correlating the clinical outcome with the TGS, thereby producing a TGS animal model.

20. The method of claim 19, further comprising classifying risk groups based on TGS.

21. The method of claim 19, further comprising:
(a) administering a therapeutic to the non-human animal,
(b) obtaining a second sample from the tumor after administering the therapeutic,
(c) determining a second TGS,
(d) comparing the first TGS and the second TGS, and
(e) correlating the change in TGS with the clinical outcome.

22. The method of claim 19, wherein the non-human animal is a mouse, a rat, a rabbit, a primate, a dog, or a pig.

23. The method of claim 19, wherein the tumor is induced, spontaneous, or transplanted.

24. The method of claim 19, wherein the species origin of the tumor differs from the non-human animal.

25. The method of claim 19, wherein determining comprises measuring LMO2 and TNFRSF9 expression by RT-PCR.

26. The method of claim 19, wherein the therapeutic is an immunomodulator.

27. The method of claim 26, wherein the immunomodulator is a chemoattractant, an antibody, a cytokine, a chemokine, or a kinase inhibitor.

28. The method of claim 27, wherein the chemoattractant is chemerin.

29. The method of claim 19, wherein the therapeutic is administered locally.

30. The method of claim 29, wherein the therapeutic is administered via intratumor injection.

31. A method of identifying a composition that lowers a TGS, the method comprising:
(a) obtaining a first sample from a tumor in a non-human animal,
(b) contacting the tumor in vivo with a composition,
(c) obtaining a second sample from the tumor,
(d) determining the TGS of the first sample and the second sample,
wherein the composition lowers the TGS of the tumor if the TGS of the second sample is less than the TGS of the first sample.

32. The method of claim 31, wherein the non-human animal is a mouse, a rat, a rabbit, a primate, a dog, or a pig.

33. The method of claim 31, wherein the tumor is induced, spontaneous, or transplanted.

34. The method of claim 31, wherein the species origin of the tumor differs from the non-human animal.

35. The method of claim 31, wherein determining comprises measuring LMO2 and TNFRSF9 expression by RT-PCR.

36. The method of claim 31, further comprising correlating TGS and clinical outcome.

37. The method of claim 31, wherein the composition is an immunomodulator.
